(19) United States
(12) Patent Application Publication
(10) Pub. No.: US 2017/0014256 A1
(43) Pub. Date: Jan. 19, 2017

(54) Method and Apparatus for Applying and Controlling Surgical Traction

(71) Applicant: Trimanus Medical Limited, Vancouver (CA)

(72) Inventors: Robert William McGraw, Vancouver (CA); John McGraw, Toronto (CA); Geoffrey Auchinleck, Vancouver (CA)

(21) Appl. No.: 14/799,823

(22) Filed: Jul. 15, 2015

(51) Int. Cl. A61F 5/042; A61F 5/37

(52) U.S. Cl. CPC A61F 5/042; A61F 5/3761

(57) ABSTRACT

Apparatus is described for applying, measuring, regulating and displaying the amount of traction force applied to a patient's lower limbs during surgery, while providing for warnings and alarms should force or time limits be exceeded.

### FIELD OF THE INVENTION

The present invention relates generally to the medical field, and more particularly to apparatus and method for applying and controlling the traction force applied to limbs during orthopaedic surgery, where traction is applied between a patient's foot or leg and a restraining post located at the patient's groin. The invention teaches applying traction force with an actuator, measuring the force applied and regulating the force applied with a control circuit in order to limit the risk of injury to the patient.

### BACKGROUND OF THE INVENTION

In the treatment of certain medical conditions it is desirable to provide a traction force to a patient's limb or limbs. Examples include distraction of the hip for hip arthroscopy or distraction of fractures of the leg bones in order to re-align the broken bones to ensure correct healing (‘fracture reduction’). In each of these cases, tension is applied to the patient's lower limb or foot. In order to maintain the traction force, it is necessary to apply a counter-force to prevent the patient's body being pulled off the operating table.

Various means have been developed to apply this counter-traction force, including harnesses and patient positions that use the patient's weight as the counter-force, however the most common approach is to provide a cylindrical post (called a ‘perineal post’) located at the patient's groin. In this position, traction force is usually applied to both the operative and non-operative limb to prevent the patient's pelvis rotating about the perineal post.

Examples of such apparatus are described in U.S. Pat. No. 1,697,121 (Knebel), U.S. Pat. No. 2,658,507 (Neufeld) and numerous others, generally called ‘fracture tables’. Variations of fracture tables provide means to apply and adjust traction forces using screw mechanisms or suspended weights (see U.S. Pat. No. 4,802,464 (Deprez) and U.S. Pat. No. 3,088,460 (Wright)).

Although this technique for applying limb traction has been in use for more than 100 years, it still presents a risk of significant injury to the patient, primarily sciatic or pudental nerve injury. Nerve injuries, largely due to the counter forces applied to the patient's groin, are reported in the medical literature at rates between 1.7% and more than 27%, most intraoperative, but many resulting in long-term damage (Telleria et al 2012, Polyois et al 2013). The risk of nerve damage is increased with increase in the applied traction force, and to a lesser extent, the length of time that traction is applied.

Traction application devices such as screws or weights can easily be (and sometimes are) equipped with indicating scales to show the amount of traction force applied. Unfortunately, such indicating scales are usually not visible during surgery as they are under the surgical drapes or otherwise obscured, and thus do not allow the surgeon to know if the traction force has increased or decreased during surgery. Furthermore, during the course of surgery, it may be necessary to alter the position of the patient, the direction or the magnitude of the traction force applied to the patient's limb in order to provide visualization of or access to the surgical site. When such changes in the direction and magnitude of the traction force are made, traditional indicating scales do not provide adequate feedback to the surgeon, nor is it possible (particularly with screw-applied traction force) to know if applied traction has increased beyond acceptable limits while the patient is repositioned. Further, should the amount of traction force need to be increased or decreased, a non-sterile surgical assistant must be called to get under the surgical drapes to adjust the applied traction force, risking contamination of the surgical field and disruption of the surgical site.

In summary, current fracture tables depend on traction applied through the lower limb, countered by a perineal post. In the surgical setting, there is no indication of the traction force applied to the operative or non-operative limb visible to the surgeon, nor is it easily possible to increase or decrease the applied traction force during the surgical procedure. Further, there is no means provided to directly measure the length of time that traction is applied to the limbs, nor is there a means to regulate the traction force applied, nor is there any provision for providing a warning to the surgeon should the traction force exceed safe limits.

The object of the current invention is to provide means for applying a selectable traction force to the patient's operative and non-operative limbs, measuring and displaying to a surgeon the traction force applied to the limbs and using a control circuit to regulate and maintain the selected amount of traction force. Further the invention includes provision of alarms to indicate if traction has exceeded safe limits, a timer to measure the duration of traction application and advantageously, a means to alert the surgeon should application of the traction force result in an unacceptably large change in position of the patient's limb.

### BRIEF DESCRIPTION OF THE DRAWINGS

The features and advantages of the present invention will become apparent upon reference to the following detailed description of the preferred embodiments and to the drawings, wherein

FIG. 1 is an illustration of a typical position for a patient on a fracture table.

FIG. 2 illustrates a mechanism for applying traction force to a patient's lower limb in accordance with the invention.

FIG. 3 is a functional block diagram of the control circuit for controlling the traction application device of FIG. 2.

FIG. 4 illustrates a typical information display as it would appear on the traction control unit.

### DETAILED DESCRIPTION OF THE PREFERRED EMBODIMENTS

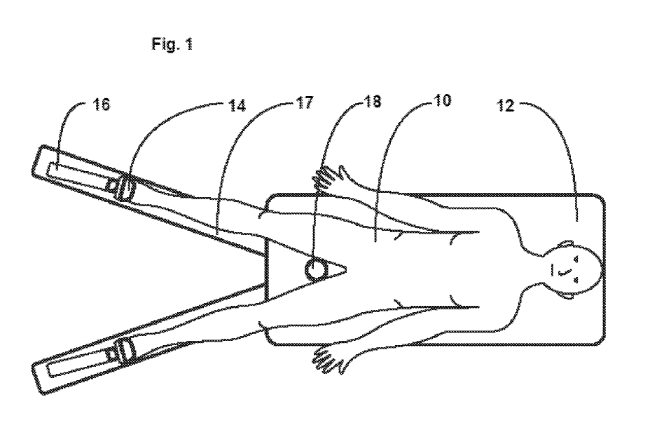

With reference now to the drawings, FIG. 1 illustrates a typical position for a patient undergoing surgery that requires traction to the lower extremity. Patient 10 is positioned in the supine position on operating table 12 which may be any one of many such tables available on the market, such as those from Maquet, Eschmann, Streris, and others. The patient's feet are put into patient applied parts 14, which in this embodiment are boot-like devices that provide a means to apply tension to the lower limb along the axis of the limb by pulling on the feet. These patient applied parts are connected to traction application devices, which are more fully described hereinafter. Traction application devices 16 are disposed to move patient applied parts 14 towards or away from the patient's body in order to increase or decrease the traction force applied to the lower limbs.

Traction application devices 16 are connected to support arms 17 which are in turn connected to the body of operating table 12 so that activating traction application device 16 will apply a force generally along the axis of the patient's leg.

To prevent patient 10 from being pulled along the surface of operating table 12 upon application of the traction force, perineal post 18 is mounted to operating table 12 and is located in the patient's groin. Perineal post 18 provides a portion of the counter force to the force applied to the patient's legs by traction application devices 16, thereby preventing the movement of patient 10's body in the direction of the applied traction force. Additional counter force is provided by the friction between the body of patient 10 and table 12. The combination of perineal post 18 and the friction force allow significantly large tension forces to be created in the lower limbs of patient 10.

It should be noted that traction could be applied to patient 10's lower limbs in other orientations using a substantially similar apparatus. For example, patient 10 could be positioned in a prone position, or in a lateral position while allowing and traction to be applied to the lower limbs in a similar manner. Further, although the current embodiment uses a boot-like apparatus to apply traction force through the feet, it is known in the art to insert a pin crosswise through the bones of the lower limb and connect this pin to traction application devices 16 to apply traction force directly to the bones of patient 10. Such variations in technique do not substantially change the function or intent of the present invention.

Returning now to the configuration of FIG. 1, perineal post 18 is usually positioned to one side or the other of the mid-line of patient 10's body. This ensures that the force applied by perineal post 18 is not applied directly to patient 10's groin, which has the potential to cause injury. Further, when applying traction force with traction application devices 16, more traction may be applied to one of patient 10's legs than the other in order to rotate patient 10's pelvis and direct the force applied by perineal post 18 away from patient 10's mid line.

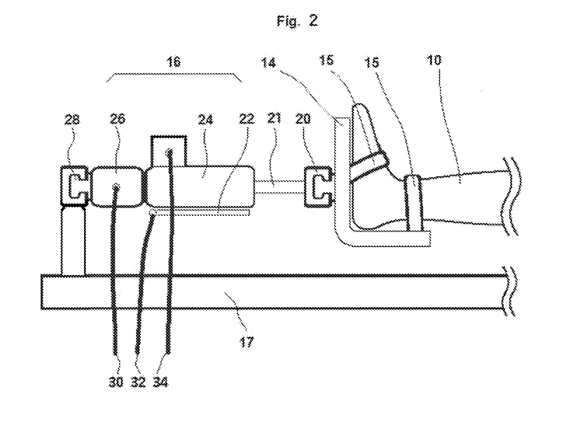

FIG. 2 is a side view of traction application device 16 according to the invention, as it would be installed for use. Traction application device includes load cell 26, which in the preferred embodiment is a Forsentek Model FL13 100 kg strain gage sensor (www.forsentek.com), electric linear actuator 24, which in the preferred embodiment is a ServoCity HDA12-20 12 volt, 180 lb thrust device which advantageously incorporates position sensor 22 and limit switches that automatically cut the power should one or the other limit of travel be reached. Finally, traction device 16 incorporates mechanical connectors 20 and 28 for mechanically affixing traction device to support arms 17 of operating table 12 and patient applied part 14. Electrical connection cables 30, 32 and 34 connect load cell 26, the motor of electric linear actuator 24 and position sensor 22 to the electronic control circuitry hereinafter described.

Mechanical connector 28 is designed to fit the accessory connector provided as part of the support arms 17 of operating table 12. Advantageously, connecter 28 can be removed and replaced with different forms of mechanical connector 28 such that traction application devices 16 can be attached to a wide range of different operating tables 12.

Similarly, mechanical connector 20 duplicates the mechanical connector provided as part of support arm 17 of operating table 12, such that patient applied part 14, which may be provided with operating table 12, can be easily connected to traction application device 16.

Once mechanically connected as shown in FIG. 2, it can be seen that linear actuator 24 can apply a traction force to the leg of patient 10 through patient applied part 14, which in this embodiment is attached to the foot of patient 10 with straps 15. By causing shaft 21 of linear actuator 24 to retract or extend, the amount of traction force can be increased or decreased. Load cell 26, being disposed between mechanical connector 28 and linear actuator 24 can measure the traction force being applied between support arm 17 and the limb of patient 10. During use, position sensor 22 measures the amount of extension of linear actuator 24.

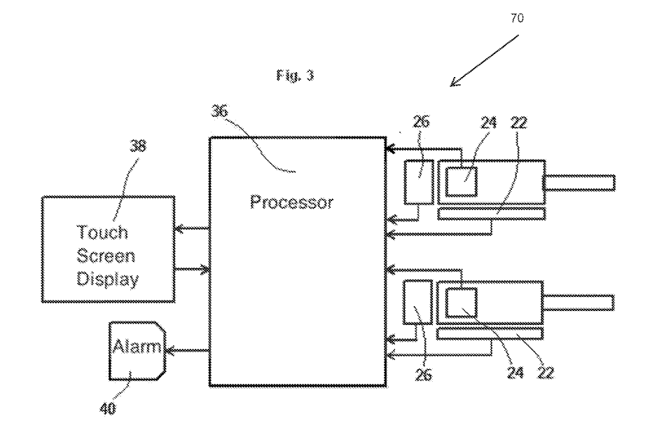

FIG. 3 is a functional block diagram of the control circuit, shown generally with reference number 70, and user controls for traction application device 16. In use, two identical traction application devices 16 are typically used—one for the each leg of patient 10, such that the amount of traction applied to each leg can be independently set and controlled as described herein.

Processor 36 is a conventional computer that includes a microprocessor containing a software program for receiving signals from load cells 26, position sensors 22 and touch screen display 38, and sending signals to linear actuator 24, touch screen display 38 and alarm 40. Processor 36 also incorporates amplifiers to properly condition the signals from load cells 26 and position sensor 22, and to provide the control current required to activate linear actuator 24.

Touch screen display 38 receives display information from processor 36 so as to provide a graphical display as hereinafter described, and sends touch information back to processor 36 in response to a user touching the various control areas of touch screen display 38 as hereinafter described.

Alarm 40 is a loudspeaker capable of reproducing audio signals produced by processor 36 and is used to produce audible alarms in the event of certain conditions being determined by the software program running on the microprocessor incorporated in processor 36.

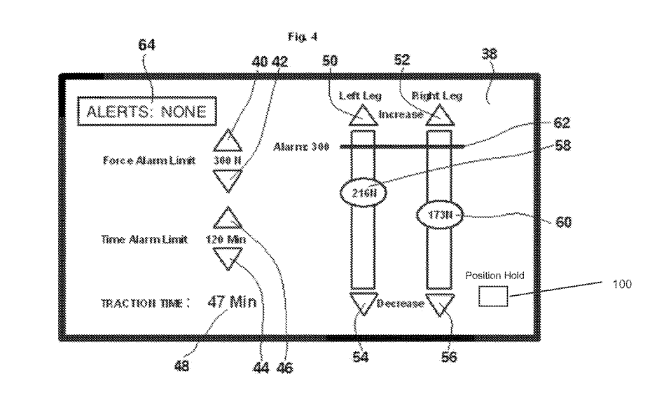

FIG. 4 shows a view of touch screen 38 as it would appear during use of the traction system. Touch screen 38 includes touch-sensitive areas that act as user controls for the traction system. Controls 40 and 42 allow the user to increase or decrease the traction force that, if exceeded as measured by load cells 26, will cause an audible alarm to sound through alarm 40. Controls 44 and 46 allow the user to increase or decrease the time limit after which an audible time limit alarm is sounded through alarm 40. Controls 50 and 54 allow the user to increase or decrease the currently desired traction force to be applied to patient 10's left leg, while display element 58 graphically represents the currently measured traction force in patient 10's left leg by its position on the vertical bar, as well as displaying the current traction force numerically. Controls and display elements 52, 56, and 60 duplicate these functions for the right leg.

In typical use, a traction application system in accordance with the invention is used as hereinafter described. Patient 10 is positioned on operating table 12, usually in a supine position. Perineal post 18 is located between the legs of patient 10 to provide counter-traction force. Support arms 17 of operating table 12 are deployed more or less along the intended axis of each leg of patient 10. Two traction application devices 16 are typically used. Each traction application device is connected to processor 36 via connection cables 30, 32 and 34. Once connected, the software running on the microprocessor within processor 36 detects the connection of the traction application devices 16 and sends a signal to linear actuator 24, causing it to fully extend. This step ensures that the maximum range of motion is available for applying traction.

Once linear actuator 24 is fully extended, each traction application device 16 is attached to connector 28 of support arm 17. In turn, patient applied part 14 is attached to traction application device 16 using connector 20. Finally the foot of patient 10 is attached to patient applied part 14 using straps 15.

Once both legs and both traction application devices 16 are connected to support arms 17, support arms 17 are extended and locked in place so that the legs of patient 10 are suspended. This will apply a small amount of traction to the legs of patient 10 due to their weight. This traction force is applied through load cell 26, hence load cell 26 is able to measure the traction force and send a force signal representing the traction force to processor 36 via connection cable 30. Processor 36 uses this force signal to calculate the traction force in standard units (either pounds force or newtons), and displays this on touch screen 38. The force signal is proportional to the force applied to the patient through the patient applied part 14.

Now that patient 10 is properly positioned, the user may use the controls on touch screen 38 to safely apply traction. As a first step, the user uses controls 40 and 42 to increase or decrease the maximum allowed traction force permitted during the current surgical procedure. Should the traction force measured by load cell 26 ever exceed this limit, processor 36 will cause alarm 40 to activate, warning the user.

Next the user sets the maximum traction application time to be allowed during the surgical procedure. The maximum time is set using touch screen controls 44 and 46. Should traction be applied for longer than this time, processor 36 will cause alarm 40 to activate, warning the user.

Now that force and time limits have been set, the user may now select the traction force to be applied to each limb. For the left limb, controls 50 and 54 are used to increase or decrease the desired amount of traction, while controls 52 and 56 are used to increase or decrease the traction applied to the right leg. Increasing the selected traction causes processor 36 to send a signal to linear actuator 24, which causes actuator 24 to retract, increasing the traction force applied to the leg of patient 10. Similarly, decreasing the selected traction causes actuator 24 to extend, reducing the traction force. Simultaneously, position sensor 22 sends a signal to processor 36 via cable 32 representative of how far linear actuator 24 is extended, that is, the position of the actuator. Using the traction force measured by load cell 26 (i.e., the force signal from the load cell), and the position measured by position sensor 22, processor 36 implements a feedback control system to regulate the traction force to be close to the desired force as set by the user. The processor 36 thus responds to the force signal from load cell 26 by generating an actuating signal that regulates the linear actuator to maintain the force applied to the patient selected part 14 at the pre-set level.

As soon as the user presses one of controls 50 or 52 to request an increase in traction force, processor 36 starts a timer, begins updating the Traction Time 48 displayed on touch screen 38, and begins checking that this time has not exceeded the maximum traction time previously set. Should the traction time exceed the time limit set with controls 44 and 46, processor 36 will cause touch screen 38 to display a warning message in alerts display 64 and will simultaneously cause alarm 40 to sound until either the traction force is reduced or control 46 is used to increase the time alarm limit.

If at any time while traction is applied, load cell 26 detects a traction force in excess of that set by controls 40 and 42, processor 36 will display a warning message in display in alerts display 64 and will simultaneously cause alarm 40 to sound until either the traction force measured by load cell 26 is reduced below the currently set level, or control 42 is used to increase the force alarm limit above the force being measured.

To further reduce the risk of injury to patient 10, the software program running on processor 36 monitors the signal from position sensor 22 and the signal from load cell 26 to ensure that traction application apparatus 16 is operating properly. Should position sensor 22 indicate that linear actuator 24 has reached its maximum or minimum extension, processor 36 will cause touch screen 38 to display a warning message in alerts display 64 and will simultaneously cause alarm 40 to sound. Similarly, should load cell 26 indicate that the traction force is significantly lower than the traction force set with controls 50, 52, 54 or 56, processor 36 will cause touch screen 38 to display a warning message in alerts display 64 and will simultaneously cause alarm 40 to sound.

During normal operation, processor 36 will cause linear actuator 24 to extend or retract by a small but predetermined amount in order to regulate the traction force as measured by load cell 26—the limits of the predetermined amount of extension or retraction being stored within processor 36. While monitoring the signal from position sensor 22, should processor 36 detect a change in position much greater than this expected small but predetermined amount, processor 36 will cause linear actuator 24 to stop moving, will cause touch screen 38 to display a warning message in alerts display 64 and will simultaneously cause alarm 40 to sound. In this way, possible injury to patient 10 due to sudden excursions of linear actuator 24 are prevented.

The current invention provides for an additional operating mode offering additional safety for patient 10. It is known that the amount of traction force required to achieve a specific clinical result, for example distraction of the hip joint for hip arthroscopy surgery, will vary over time. Usually a higher level of traction force is initially required to achieve a desired amount of distraction, but the force required decreases over time as the tissues resisting the traction force relax. In this situation, it is advantageous to allow the traction force applied to decrease as long as the amount of distraction is maintained. In this alternate mode of operation according to the invention, the user will use controls 50 and 52 to initially set a traction force that attains the degree of distraction required. Once the clinically required level of distraction is attained, the user would then use an additional control 100 (shown in FIG. 4) to cause processor 36 to switch from force regulating mode to position regulating or position hold mode. In position regulating mode, processor 36 will use the signal from position sensor 22 to determine the target position to be maintained, and send signals to linear actuator 24 to ensure that it stays in the same position as the traction force measured by load cell 26 decreases. In practice, this ensures that the minimum force required to attain the desired clinical result is used, decreasing the risk of injury to patient 10.

Many other variations to the apparatus described herein may be constructed without departing from the scope and spirit of the present invention. For example, although the preferred embodiment uses wired connections between processor 36 and traction application apparatus 16, traction apparatus 16 could be equipped with a battery power supply, a local microprocessor and a wireless connection to processor 36; additional controls could be added to touch screen 38 such as an alarm silence control to temporarily mute alarm 40; or an additional display element on touch screen 38 could show the current amount of extension of linear actuator 24. As another example of an equivalent alternative structure, the special arrangement of the load cell 26, linear actuator 24 and patient applied part 14 shown in FIG. 2 may be reversed so that the load cell is attached to the patient applied part and the linear actuator is attached to the connector 28. In either orientation, operation of the linear actuator applies traction to the patient's limb.

While the present invention has been described in terms of a preferred embodiment, it will be appreciated by one of ordinary skill that the spirit and scope of the invention is not limited to those embodiments, but extend to the various modifications and equivalents as defined in the appended claims.

We claim:

1. A surgical traction device, comprising:
a linear actuator having an extendable and retractable shaft, the linear actuator configured for applying a traction force through the shaft in response to an actuating signal;
a load cell adapted to generate a force signal that is proportional to the force applied to the patient applied part by the linear actuator;
a controller adapted to receive the force signal from the load cell and in response thereto generate the actuating signal;
wherein, said actuating signal causes said linear actuator to extend or retract in order to maintain the force applied to said patient applied force at a pre-selected level.

2. The surgical traction device according to claim 1 including a position sensor for determine the position of the linear actuator.

3. The surgical traction device according to claim 2 including a patient applied part connected to the shaft, and wherein the position of the linear actuator and the force signal are used to maintain the force applied to the patient at the pre-selected level.

4. The surgical traction device according to claim 3 in which the patient applied part is attached to a patient's lower limb and wherein the load cell is attached to an anchor point that is fixed with respect to the patient's lower limb so that the linear actuator may apply force between the anchor point and the patient applied part.

5. The surgical traction device according to claim 4 wherein a predetermined maximum and minimum force value for the force applied to the patient applied part may be set in the controller and wherein if the maximum or minimum force value is exceeded an alarm is activated.

6. The surgical traction device according to claim 5 wherein a predetermined maximum and minimum time value for the duration of time during which application of force may be applied to the patient applied part may be set in the controller and wherein if the maximum or minimum time value is exceeded an alarm is activated.

7. A surgical traction device, comprising:
a linear actuator responsive to actuating signals and adapted to apply traction force to a patient applied part;
a force measurer connected to the linear actuator for generating a force signal that is proportional to the force applied by the linear actuator to the patient applied part; and
a control circuit configured to receive the force signal and produce the actuating signals so as to cause the linear actuator to move so as to maintain the force signal at a pre-selected level.

8. The surgical traction device of claim 7 in which the patient applied part is connected to a first end of the linear actuator and a second end of the linear actuator is fixed to an anchor point with respect to the patient's lower limb such that the linear actuator can apply a traction force between the anchor point and the patient applied part.

9. The surgical traction device of claim 7 further comprising a display for displaying the force signal measured by the force measuring means.

10. The surgical traction device of claim 7 further comprising input means for selecting a pre-selected force signal level.

11. The surgical traction device of claim 7 further comprising an alarm for providing an alarm signal should the force signal differ from the pre-selected force signal level.

12. The surgical traction device of claim 9 further comprising input means for selecting a pre-selected time value for the duration of time that traction has been applied.

13. The surgical traction device of claim 12 further comprising an alarm for providing an alarm signal should the pre-selected time value differ from the pre-selected time value.

14. A surgical traction device, comprising:
a linear actuator responsive to actuating signals and having an extendable and retractable linear actuator shaft interconnected to a patient's limb;
a load cell connected to the linear actuator for producing a force signal indicative of the force applied by the linear actuator to the patient's limb;
a position sensor for generating a position signal indicating a position of the linear actuator shaft;
a control circuit for receiving the position signal and the force signal and for producing the actuating signals in response to the position and force signals; and
an input for selecting a desired force, wherein the control circuit produces actuating signals to cause the linear actuator to move the shaft such that the force signal is equal to the desired force.

15. The surgical traction device as in claim 14 further comprising a display for displaying the force signal.

16. The surgical traction device as in claim 15 further comprising an alarm for alerting a user should the force signal differ from the desired force.

17. The surgical traction device as in claim 14 further comprising a display for displaying the position signal.

18. The surgical traction device as in claim 14, wherein once the control circuit has produce actuating signals to move the linear actuator shaft so as to make the force signal equal the desired force, the control circuit then produces actuating signals to keep the position signal constant.
